(19) United States
(12) Patent Application Publication
(10) Pub. No.: US 2026/0346457 A1
(43) Pub. Date: Nov. 5, 2026

(54) Cobot-Based Patient Care for Biometric Monitoring and Postoperative Rehabilitation Support

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/430,366

(22) Filed: Dec. 23, 2025

Related U.S. Application Data
(63) Continuation-in-part of application No. 19/195,731, filed on Apr. 30, 2025.

(51) Int. Cl. B25J 9/16; A61B 5/00; A61B 5/0205; A61B 5/11; A61B 5/1171; A61H 1/02; A61B 5/021; A61B 5/024; A61B 5/08

(52) U.S. Cl. CPC B25J 9/1694; A61B 5/0002; A61B 5/02055; A61B 5/1126; A61B 5/1176; A61B 5/746; A61H 1/02; A61B 5/021; A61B 5/024; A61B 5/0816; A61B 2562/0219; A61H 2201/0157; A61H 2201/1215; A61H 2201/5053; A61H 2201/5084; A61H 2230/42; A61H 2230/50

(57) ABSTRACT

A collaborative robotic system is provided for postoperative patient care, combining autonomous mobility, biometric monitoring, robotic-assisted rehabilitation, and clinical data integration. The system includes a mobile cobot configured to navigate to a patient’s bedside, identify the patient, and collect real-time biometric data using integrated sensors. A robotic manipulator guides the patient through rehabilitative exercises, with assistance levels adjusted by an adaptive control system based on biometric feedback. A machine learning predictive analytics module analyzes patient data to predict recovery trajectories and identify complications. An interactive communication interface delivers exercise instructions and health updates through visual, auditory, or haptic feedback. The system wirelessly transmits patient data to electronic health records and incorporates safety and compliance protocols including collision detection, privacy safeguards, and regulatory adherence. The system enables personalized, data-driven postoperative care and supports continuous monitoring, remote oversight, and automated rehabilitation intervention.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by reference.

### TECHNICAL FIELD

The present invention relates generally to collaborative robotic systems for healthcare applications and, more particularly, to a cobotic platform configured for postoperative patient support.

### BACKGROUND

Postoperative recovery in hospital and outpatient settings involves a multifaceted care regimen that includes vital sign monitoring, physical rehabilitation, patient engagement, and clinician oversight. The quality and continuity of this care directly affect patient outcomes, rates of readmission, and long-term functional recovery. However, the growing demand on clinical staff, variability in patient responsiveness, and limitations of traditional rehabilitation protocols pose challenges to consistently delivering high-quality, individualized postoperative care.

Existing postoperative care practices typically rely on intermittent monitoring of biometric data using separate vital sign monitors, while physical therapy is administered manually by clinical staff or delayed until formal sessions with therapists. These approaches can result in missed early indicators of complications, limited data to assess patient progress, and inconsistent rehabilitation intensity. Moreover, manual tracking of therapy performance and recovery trajectories often lacks the granularity and objectivity needed to support dynamic, data-driven care decisions.

Recent advances in robotics and artificial intelligence offer an opportunity to address these gaps. Collaborative robots, or cobots, equipped with biometric sensors and rehabilitation capabilities, may provide autonomous bedside interaction, personalized therapy delivery, and real-time data acquisition. Such systems could empower clinicians with continuous visibility into patient status and enable early intervention when deviations from expected recovery patterns occur.

Despite promising technological advances, current robotic systems lack integration across the domains of mobility, sensing, actuation, adaptive control, and clinical interoperability. There remains a need for a cohesive cobotic platform that can safely interact with patients in a postoperative setting, assist with personalized physical therapy exercises, monitor biometric signals in real time, adapt its operation dynamically based on patient-specific data, and seamlessly integrate with hospital information systems to support longitudinal care planning.

### SUMMARY

The present disclosure relates to a collaborative robotic system for postoperative patient care, integrating autonomous mobility, biometric monitoring, robotic-assisted rehabilitation, and clinical data communication capabilities. The system includes a mobile cobot platform configured to navigate hospital or outpatient environments and interact with patients at bedside or in rehabilitation areas. An integrated biometric monitoring suite continuously collects patient vital signs, including heart rate, respiratory rate, temperature, blood pressure, and motion data, using a combination of non-invasive sensors.

A robotic rehabilitation module assists the patient in performing prescribed physical therapy exercises. The assistance provided by the robotic manipulator is dynamically adjusted by a control unit based on real-time biometric feedback and patient-specific recovery metrics. The control unit implements an adaptive control system and may further incorporate a machine learning model to predict patient recovery trajectories, detect early signs of complications, and recommend adjustments to the rehabilitation protocol.

The system further includes a patient identification module 260, such as an RFID reader or facial recognition system, to retrieve and execute a personalized care plan for each patient. An interactive communication interface, featuring visual, auditory, or haptic feedback, provides exercise instructions, progress updates, and engagement prompts to the patient. A wireless communication interface enables the system to transmit patient data securely to a centralized healthcare information system, supporting remote monitoring, care coordination, and compliance tracking.

Safety features include collision detection, adherence to sterile and privacy boundaries, and operational constraints to ensure regulatory compliance and patient protection. The system may operate in real time, adjusting therapeutic interventions as patient status evolves, and enabling data-driven, individualized recovery support in both inpatient and outpatient settings.

### BRIEF DESCRIPTION OF DRAWINGS

The foregoing and other objects, aspects, features, and advantages of the disclosure will become more apparent and better understood by referring to the following description taken in conjunction with the accompanying drawings, in which:

FIG. 1 is a perspective view of a collaborative robotic system for biometric monitoring and rehabilitation support.

FIG. 2 is a block diagram of the system architecture.

FIG. 3 is a schematic diagram illustrating biometric data acquisition from a patient.

FIG. 4 is a flowchart illustrating real-time adaptive control process for dynamically adjusting rehabilitation assistance based on biometric feedback.

FIG. 5 is a block diagram of a machine learning analytics module.

FIG. 6 is a schematic depiction of an interactive communication interface.

FIG. 7 is a communication diagram showing data transmission from a cobotic system to external systems.

FIG. 8 is a flowchart depicting a method of operation for the collaborative robot.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

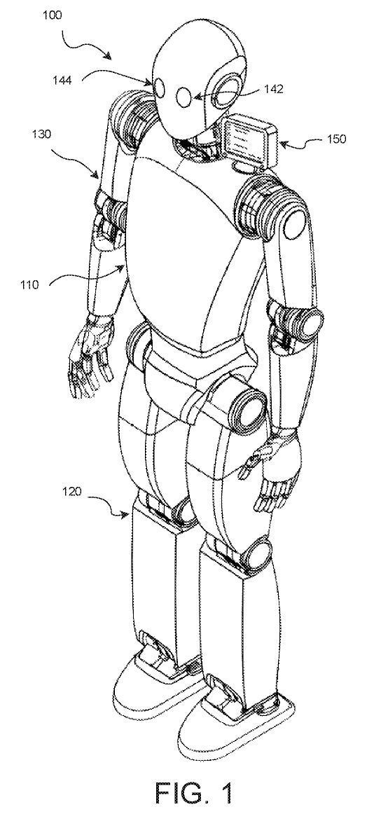

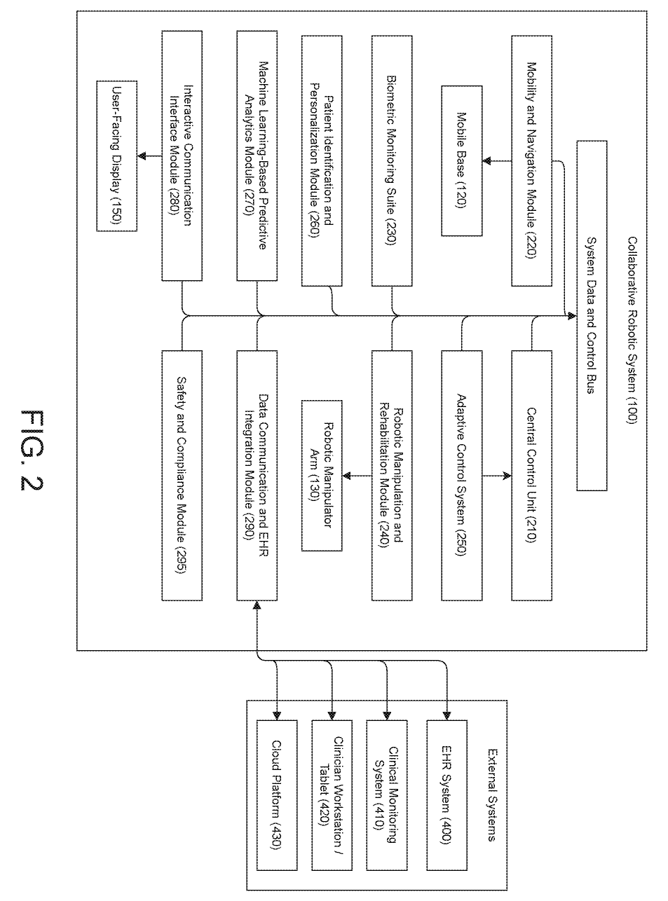

FIG. 1 illustrates an embodiment of a collaborative robotic system 100 for patient care, biometric monitoring, and rehabilitation support. The system 100 is a mobile cobot (collaborative robot) that includes a mobile base 120 for autonomous navigation, at least one robotic manipulator arm 130, and a user-facing display 150. In the depicted configuration the mobile base 120 is bipedal. In other configurations, the mobile base 120 houses motors and wheels enabling the cobot 100 to move around a hospital or clinic room and position itself next to a patient’s bedside. The base 120 also carries on-board power (e.g., rechargeable battery) and sensors for mapping and obstacle detection so that the robot can safely navigate without collisions in a dynamic environment. Mounted on the base 120 is a torso 110 or frame supporting the robotic arm 130 and the display 150. The robotic manipulator 130 is capable of human-like articulation to assist with physical therapy exercises, having multiple degrees of freedom to mimic a human arm’s range of motion. For example, the manipulator 130 can gently grasp or support a patient’s limb during rehabilitative movements. Integrated force/pressure sensors and optical encoders in the arm 130 provide feedback to the control system 250 (shown in FIG. 2), ensuring that the cobot’s physical interactions remain safe and do not exert excessive force on the patient. An emergency stop or safety override may be included to immediately halt motion if unsafe conditions are detected, further safeguarding the patient.

The cobot 100 carries an integrated biometric monitoring suite 230 for continuously measuring the patient’s vital signs. The system includes sensors to capture heart rate, respiratory rate, body temperature, blood pressure, and patient motion/activity, among other metrics. Some sensors may be built into the robot (for example, an infrared temperature sensor or camera for monitoring breathing and pulse), while others may be wearable devices or attachments applied to the patient. In one embodiment, the cobot 100 uses facial recognition or scans an RFID wristband to identify the patient upon approach, then automatically retrieves that patient’s medical profile for personalized care. Once the patient is identified, the robot can apply or activate appropriate biometric sensors, for instance, gently wrapping a wireless blood pressure cuff around the patient’s arm, placing a finger clip pulse oximeter, or initiating a contactless vital sign measurement. The system 100 is designed to accommodate non-invasive monitoring; it can utilize instruments such as electronic stethoscopes, thermometers, SpO₂ sensors, ECG electrodes, or even camera-based vital sign detection. In some implementations, the robot employs thermal imaging and RGB cameras to measure vital signs like temperature, heart rate, and breathing without any direct contact. Collected vital data (e.g., oxygen saturation, blood pressure, heart rate, respiratory rate) is streamed in real-time to the cobot’s onboard computer for analysis and can also be relayed to healthcare staff as needed. The biometric monitoring suite thus enables continuous observation of the patient’s physiological state during recovery.

The system’s user-facing display 150 and communication interface 280 (shown in FIG. 2) provides an interactive portal for both the patient and healthcare providers. In this embodiment, a touch-screen display 150 is mounted at roughly eye-level to the patient (for a humanoid form, this may be on the “head” or chest of the cobot 100) so that it can present visual information, such as the patient’s exercise instructions, vital sign readouts, or progress dashboards. The cobot 100 also includes audio capabilities: speakers for verbal output (e.g., coaching the patient through an exercise or providing reassurance) and microphones to capture the patient’s speech or distress calls. This multi-modal interface allows the cobot 100 to engage in two-way communication, for example, the robot can ask the patient questions about pain levels or understanding of instructions, and the patient can respond verbally or via the touch-screen. The interface is designed to be user-friendly and supportive, it can provide clear visual prompts, auditory cues, or gentle haptic feedback to guide the patient through rehabilitation activities and keep them informed of their progress. In addition, the cobot 100 is equipped with connectivity for telepresence and remote communication. A high-resolution camera 142 on the robot may enable remote healthcare providers (such as a nurse or doctor) to have a live video interaction with the patient through the robot’s display 150. The cobot 100 also includes an infrared sensor unit 144 to collect additional environmental and biometric data. This feature allows a nurse to “virtually enter” the patient’s room via the cobot 100, converse with the patient, and review the sensor data in real time as if present at the bedside. The cobot’s communication hardware supports wireless networking (for instance, built-in antennas for Wi-Fi and 5G) so that it can transmit and receive data securely over the hospital network. Through this interface, the system not only serves as a rehabilitation coach but also as a communication hub linking patients with their care team and providing comfort and companionship during recovery.

It should be noted that while FIG. 1 depicts a humanoid cobot embodiment (with a human-like form factor for the manipulator 130 and interface placement), the invention is not limited to this configuration. In alternate embodiments, the collaborative robot could take a non-humanoid or more modular form, for example, a wheeled cart or bedside table form with an articulating robotic arm 130 and sensor mast, or a smaller module that can attach to a standard hospital bed. The modular design enables task-specific reconfiguration; the cobot’s external shell and appearance can be adapted based on patient age or use-case, presenting a friendly avatar for pediatric patients or a streamlined clinical design for adults. All such variations include the core components shown in FIG. 1 (mobile base 120, sensor suite, manipulator 130, and interface 150) and are designed to perform equivalent functions in providing autonomous patient monitoring and rehabilitation assistance.

FIG. 2 is a block diagram illustrating the system architecture of the cobot-based patient care and rehabilitation system. The architecture is organized into functional modules that work together under a central control unit 210. The primary components and modules include:

Central Control unit 210: One or more processors (and associated memory) that execute the control software and algorithms for the cobot 100. This module governs overall operation, data processing, and coordination of the other subsystems. The control unit 210 runs the cobot’s operating system and high-level decision-making routines, including the adaptive therapy logic and predictive analytics described later. In practice, the control unit 210 may be a combination of onboard microcontrollers, CPUs/GPUs, and possibly cloud-connected computing resources 430, all configured to act in concert as the “brain” of the system.

Mobility and Navigation module 220: This module controls the mobile base 120 and locomotion of the cobot. It includes motors, wheel drive assemblies or other locomotion means, and associated motor drivers. It also integrates navigation sensors such as LiDAR, ultrasonic sensors, depth cameras, or similar, which allow the cobot to map its environment and detect obstacles. A navigation controller within this module plans paths to move the cobot autonomously to a target location (e.g., navigating from a charging station to a patient’s bedside) while avoiding collisions. The mobility module 220 may implement simultaneous localization and mapping (SLAM) algorithms and incorporate safety features like proximity sensors and bump sensors for real-time obstacle avoidance.

Biometric Monitoring Module 230: This module 230 represents the integrated vital sign sensor suite of the system. It comprises the various sensors that acquire patient biometric data, including heart rate monitors, respiratory rate sensors, temperature sensors, non-invasive blood pressure sensors (cuffs), pulse oximeters for blood oxygen levels, and motion or activity sensors. These sensors can be embedded on the robot (such as built-in cameras or IR thermometers) or wirelessly paired/wearable devices on the patient’s body. The biometric module conditions and digitizes the sensor signals and streams the data to the control unit 210 for real-time monitoring. It may also include analog front-end circuits for physiological sensors (e.g., ECG amplifiers) and ensure that measurements are taken at clinically relevant intervals or continuously as required.

Robotic Manipulation & Rehabilitation module 240: This module encompasses the hardware and control systems for the robot’s manipulator arm 130 and any rehabilitation tools (such as grasping end-effectors or attachments for exercise assistance). It drives the joints of the robotic arm 130 and controls its interactions with the patient. The module is configured to provide robotic-assisted physical therapy, meaning it can guide the patient’s limbs through prescribed rehabilitative movements or exercises. Actuators (motors or hydraulic/pneumatic drives) in each joint are controlled to move along defined trajectories or to apply supportive forces during an exercise. The rehabilitation module 240 also monitors forces/torques via sensors on the arm 130 to detect patient effort or resistance, enabling adjustable assistance levels. For example, if a patient can initiate a movement but not complete it, the controller will sense the effort and apply just enough force to help finish the motion, thereby practicing an “assist-as-needed” paradigm. This module works closely with the adaptive control system 250 described below to modulate its output based on patient performance.

Adaptive Control System 250: This is the real-time feedback control module responsible for adjusting therapy parameters on the fly. The adaptive control system 250 receives input from the biometric sensors and the manipulator 130 (e.g., vital signs, patient motion, exerted forces) and uses that data to dynamically adjust the rehabilitation protocol. For instance, it can modify the speed, range, or resistance of an exercise in response to the patient’s condition, if the patient’s heart rate rises above a threshold or signs of fatigue are detected, the control might slow down or pause the activity; conversely, if the patient is performing well, the system may increase the challenge slightly to promote improvement. The adaptive controller follows predefined clinical rules and safety criteria (e.g., maximum allowed heart rate or pain level) in making these adjustments, ensuring that therapy stays within safe limits while remaining effective. This module effectively implements a closed-loop control loop for rehabilitation, tailoring the robot’s assistance in real time to the patient’s needs and recovery goals.

Patient Identification And Personalization Module: This module handles recognizing individual patients and customizing the system’s operation to each patient’s profile. It may include a camera with facial recognition software and/or an RFID or barcode reader for scanning patient ID tags. Upon identifying the patient, the system can retrieve personal health data such as the patient’s medical history, surgery details, or prescribed rehab regimen from an internal database or the hospital’s electronic records. Using this information, the cobot personalizes its monitoring thresholds and therapy settings, for example, adjusting target exercise parameters based on the patient’s age, baseline fitness, or specific recovery plan. This module ensures that care is tailored to “the individual patient profile,” meaning the device adapts to differences in patient size, ability, and medical needs rather than providing a one-size-fits-all treatment. It also maintains patient-specific data security, ensuring that any retrieved personal information is handled according to privacy regulations.

Machine Learning-Based Predictive Analytics Module 270: This module 270 analyzes the biometric and activity data collected over time to forecast the patient’s recovery trajectory and to detect any early signs of complications. It leverages machine learning algorithms, which may run on the cobot’s processor or on a connected cloud platform 430, to process patterns in vital signs, patient compliance, and performance trends. Using these data, the predictive analytics module 270 can predict patient recovery outcomes, such as estimating the likely improvement in mobility by a certain date, or identifying if the patient is at risk of a postoperative complication (e.g., an infection or cardiopulmonary issue) before it becomes acute. For example, a significant deviation in certain vital sign patterns or rehab progress metrics could be flagged by the ML model as correlating with potential setbacks. The module can generate alerts or recommendations based on its predictions, for instance, suggesting modifications to the therapy plan or prompting a clinician’s evaluation if a risk threshold is crossed. Over time, as more data is gathered, the machine learning model can continuously update/retrain to improve its predictive accuracy for the specific patient and across populations.

Interactive Communication Interface Module 280: This module corresponds to the patient-facing and caregiver-facing interface components of the system. It includes the touch-screen display 150, audio subsystem (speakers and microphone), any indicator lights or haptic feedback devices, and the user interface software. Through this module, the system provides user-friendly visual, auditory, and haptic feedback to the patient, for example, displaying simple exercise instructions or progress graphs on the screen, speaking encouragement or warnings, and even vibrating or gently tapping via the robotic arm 130 to get the patient’s attention if needed. It also manages input from the patient, such as button presses on the screen or voice commands (if voice recognition is enabled). Importantly, the communication interface module 280 connects the patient with remote healthcare providers: it may incorporate telepresence features allowing video or audio calls through the cobot, effectively turning the robot into a two-way communication node in the hospital’s network. This module is designed with accessibility in mind, ensuring that patients with limited mobility or tech experience can easily interact with the cobot (large, clear on-screen buttons, multilingual support, etc.). It also handles any clinical notifications, such as sounding an alarm if an urgent vital sign abnormality is detected.

Data Communication and EHR integration module 290: This module 290 enables the system to wirelessly transmit patient data and device logs to external systems, such as electronic health records (EHR) 400 databases, hospital information systems, or clinician workstations. It encompasses the network interfaces (Wi-Fi, Bluetooth, 4G/5G cellular, etc.) and communication protocols needed for secure data exchange. For example, this module can send the patient’s vital statistics and exercise results at regular intervals to the hospital’s central monitoring system or directly record them into the patient’s digital chart in the EHR 400. It can also receive data, such as updated therapy orders or medication adjustments from the EHR 400. Ensuring data integrity and privacy is a key function of this module: all transmissions may be encrypted and sent over secure channels, and the module is designed to comply with healthcare data standards and regulations (e.g., HL7/FHIR for data format, HIPAA for privacy in the US). This integration allows the cobot to function as part of the larger clinical IT ecosystem, for instance, nurses and doctors can review the cobot-collected data from within their usual patient record software, and any alerts generated by the cobot can be logged for compliance and follow-up.

Safety and Compliance Module 295: Overseeing all aspects of the system is the safety and compliance module 295, which implements the safety protocols, failsafes, and regulatory compliance features of the cobot. This includes hardware safety mechanisms like emergency stop buttons and torque limiters, as well as software routines that continuously monitor for unsafe conditions. The module’s functions include collision detection and avoidance, using the cobot’s sensors to detect people or objects in its path and stopping or rerouting as needed to prevent collisions. It also enforces limitations on the robot’s speed and force when near a patient or caregiver. For example, if the robot’s arm 130 encounters unexpected resistance (indicating it may be pushing against the patient or an obstacle), the safety controller halts movement immediately. Patient privacy safeguards are also managed here, ensuring that camera feeds or data are not stored or transmitted without authorization and that all patient-specific information is handled according to privacy guidelines. Additionally, the module ensures compliance with medical device regulations and standards (for example, IEC 60601 for electrical safety, ISO 13482 for safety of personal care robots, etc.). It performs self-diagnostics and can prompt scheduled maintenance or calibration to keep the system operating within safe parameters. This safety and compliance module 295 thus provides a critical layer of oversight, allowing the cobot to operate autonomously around vulnerable patients with a high degree of trust and reliability.

All the above modules are operatively connected and coordinated by the central control unit 210 to function as an integrated whole. It will be understood by those skilled in the art that each module can be implemented in hardware, software, or a combination of both. For example, the control and analysis functions described (adaptive control algorithms, predictive analytics, communication protocols) may be carried out by software programs executed on the cobot’s processors, and those processors can be any form of computing device or circuitry suitable for data processing. The modular breakdown in FIG. 2 is logical; physical implementations may combine or distribute these functions across multiple physical devices or cloud services 430 as long as the overall functionality remains as described. In some cases, certain modules (e.g., the predictive analytics) can run on a remote server or cloud platform 430, with the cobot transmitting data to that server and receiving back results. The design is flexible to accommodate different deployment scenarios (fully self-contained robot vs. networked robot) while still providing all the features needed to monitor patient biometrics and support rehabilitation in a safe, adaptive, and intelligent manner.

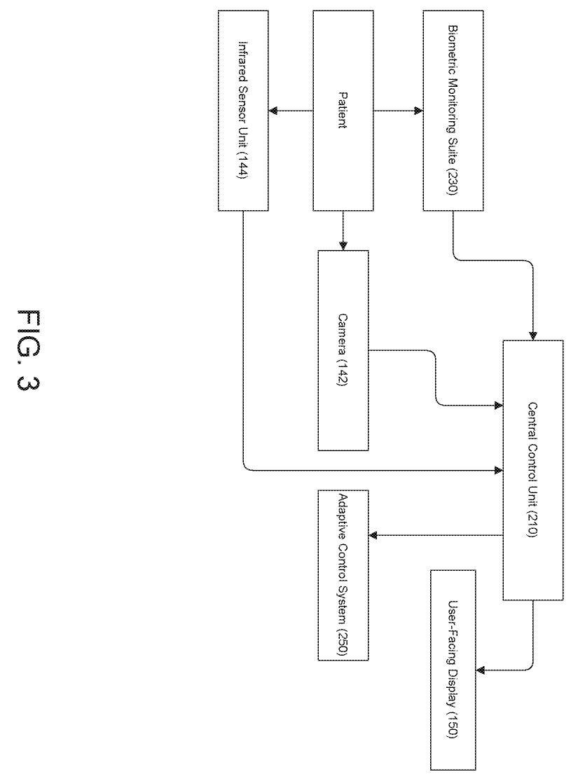

FIG. 3 depicts the process of biometric data acquisition from the patient by the cobot system. In this illustration, the cobot 100 is positioned at the patient’s bedside and is initiating the collection of vital signs and other biometric indicators. The figure shows various sensors in use, both contact-based and contactless, to gather a comprehensive set of patient data.

In one scenario, after verifying the patient’s identity (using the methods described above, such as facial recognition or scanning an ID tag), the cobot applies the necessary biometric sensors to the patient. For example, the cobot could gently place a blood pressure cuff on the patient’s arm via its robotic hand, or attach electrode pads to the patient’s chest for heart rate/ECG monitoring. Similarly, the cobot could perform contactless sensing, e.g. the cobot’s camera 142 and infrared sensor unit 144 can be directed toward the patient to measure respiratory rate (by analyzing chest movements or thermal fluctuations at the nostrils) and skin temperature (via IR thermography) without touching the patient. Simultaneously, a fingertip pulse oximeter sensor (either held by the robot or worn by the patient) measures blood oxygen saturation and pulse. The cobot’s central control unit 210 aggregates these signals in real time.

The biometric monitoring suite 230 is capable of capturing both baseline vital signs and continuous data streams. For instance, at the start of a rehabilitation session or during postoperative rounds, the cobot can perform a quick assessment capturing temperature, blood pressure, heart rate, and oxygen saturation all within a minute or two, similar to a nurse’s routine vital check. The system leverages a variety of medical-grade sensors to do so: thermometers (either IR non-contact or an ear/skin thermometer as an attachment on the robot’s hand), blood pressure sensors (an automatic oscillometric cuff that the robot can inflate/deflate), heart rate and cardiac rhythm sensors (which could be derived from a chest ECG or from a PPG sensor in a finger clip), respiratory rate sensors (such as a respiratory inductance plethysmography band or camera-based motion detection), and activity or motion sensors (accelerometers or gyroscopes that track the patient’s movements). The cobot may also use specialized tools like a digital stethoscope to listen to heart and lung sounds, or a glucometer to measure blood glucose if needed for certain patients, FIG. 3 generically represents these possibilities with a patient interface for additional sensors (e.g., a port or tray on the cobot holding such instruments). In one embodiment, the cobot places wearable devices on the patient: for example, a smart wristband for continuous heart rate and sleep monitoring, or adhesive wireless patches that stream ECG and respiratory data. These wearables communicate with the cobot via Bluetooth or other wireless protocols as part of the biometric module.

Crucially, the biometric data acquisition is designed to be non-invasive and patient-friendly. The cobot’s arm 130 performs gentle, precise motions when applying sensors, informed by force feedback to avoid discomfort. The system can explain to the patient each step via the display 150 or voice (“Now measuring your blood pressure, please remain still...”). Because the cobot can operate autonomously, measurements can be taken at scheduled intervals through day and night without always requiring a human nurse to be present. For example, the system could be programmed to take vital sign readings every 15 minutes post-surgery and alert staff if any reading is outside preset limits. The data acquired is immediately processed and visualized by the cobot, e.g. the cobot 100 might show a small panel on the display 150 with real-time waveforms or numeric readouts of the vitals. This immediate feedback allows both patient and any nearby caregiver to observe the readings.

The biometric data acquisition process illustrated in FIG. 3 is comprehensive. As one example of its capabilities, consider a postoperative patient who needs both monitoring and assistance: the cobot can simultaneously measure ECG (heart electrical activity), SpO₂ (oxygen saturation), skin temperature, and blood pressure, essentially replicating the functions of standard vital sign monitors. In addition, because the cobot is a mobile and intelligent platform, it can measure contextual data such as the patient’s position (are they lying down or sitting up, determined via vision or an IMU) and activity level (how much the patient has moved around today). All these data points together form the patient’s biometric profile, which the system records and analyzes. As indicated by FIG. 3 the data flows from the sensors back to the cobot’s control system 250, where it is stored and evaluated. The system may filter and clean the raw data (e.g., remove motion artifacts from heart rate signals) and then use it for both immediate decision-making and longer-term trend analysis.

Another aspect shown in FIG. 3 is the cobot’s ability to assist with patient mobility while monitoring. For instance, if part of rehabilitation involves the patient practicing standing or walking, the cobot can physically support the patient under their arm or with a gait belt. As it does so, its sensors continue to record vitals like heart rate and blood pressure response to exercise, as well as the patient’s balance and gait characteristics. This dual capability of monitoring during active therapy is a key advantage. In some embodiments, the cobot can function similarly to a smart walker or therapy assistant, helping a patient walk across the room while keeping track of their stability and vital signs. Studies have shown the feasibility of robots assisting in gait training for home or clinical rehab, and here the cobot serves that role by integrating the support function with real-time vital monitoring. If the patient becomes unstable or vitals change abruptly, the cobot can detect this immediately and respond (e.g., stop the activity or signal for help).

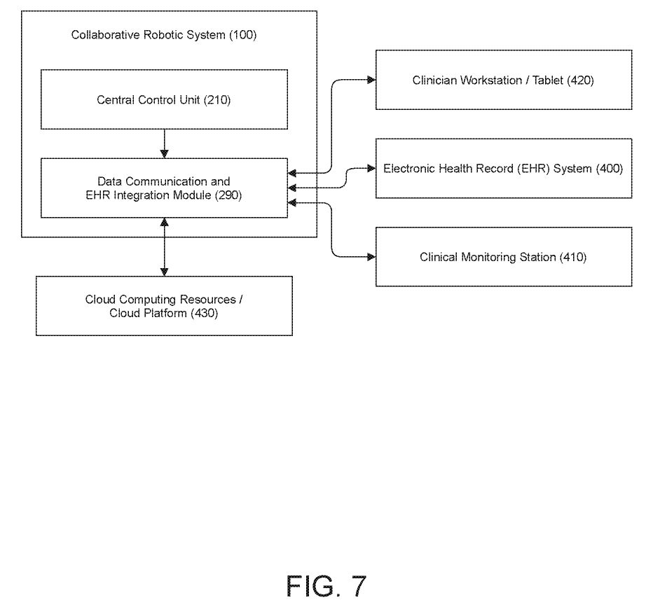

All biometric data acquired in FIG. 3’s process is time-stamped and saved to the system’s memory. It is also prepared for transmission to external systems (as detailed in FIG. 7). By collecting comprehensive biometric information in a seamless, automated way, the cobot system ensures that no subtle sign of patient distress or progress is missed. This continuous monitoring underlies the adaptive control and predictive analytics described in subsequent figures.

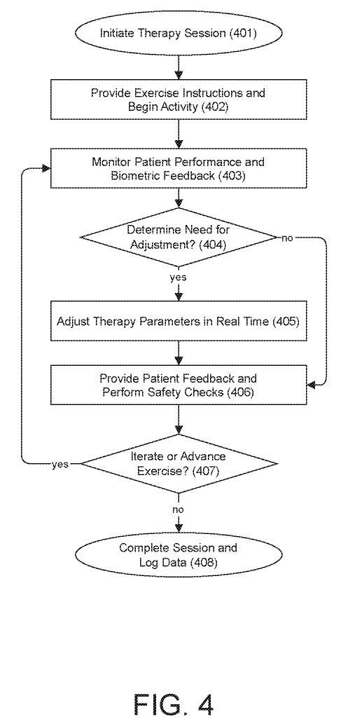

FIG. 4 is a flowchart illustrating the adaptive control logic of the cobot’s rehabilitation support system. The flowchart depicts a closed-loop process by which the system dynamically adjusts rehabilitation assistance in real time based on patient-specific biometric feedback, performance metrics, and predefined clinical and safety criteria. The adaptive control process is implemented by the control unit 210 and the adaptive control system 250 and proceeds through the following numbered steps.

Initiate Therapy Session (Step 401). The adaptive control process begins by initiating a rehabilitation therapy session. In Step 401, the cobot identifies the patient and obtains baseline biometric data as described with respect to FIG. 3. The system retrieves the patient’s prescribed rehabilitation plan, including the type of exercises, target repetitions, range-of-motion goals, and initial intensity or assistance levels. The cobot positions its robotic manipulator 130 and prepares any required apparatus to assist the patient’s limb. Initial therapy parameters, such as assistance force, movement speed, and motion limits, are established based on the patient’s profile. A preliminary safety check is performed to confirm that the patient’s vital signs are within acceptable ranges and that the patient is ready to proceed.

Provide Exercise Instructions and Begin Activity (Step 402). In Step 402, the cobot provides exercise instructions to the patient via the interactive communication interface, including visual prompts on the user-facing display 150 and/or verbal guidance. The system then initiates the rehabilitative activity, which may include active exercises, passive range-of-motion movements, or assisted movements. The adaptive control system 250 sets an initial movement trajectory, force output, or resistance level in accordance with the rehabilitation plan. During this step, the system continuously measures performance data, including joint angles, applied forces, motion smoothness, and concurrent biometric signals, which are supplied as inputs to the adaptive control logic.

Monitor Patient Performance and Biometric Feedback (Step 403). During execution of the exercise, the system performs continuous monitoring in Step 403. Biometric feedback, such as heart rate, blood pressure, respiratory rate, and indicators of fatigue or discomfort, is acquired by the biometric monitoring suite 230. Performance feedback, including patient-applied force, achieved range of motion, and movement consistency, is also collected. The control unit 210 evaluates these inputs relative to historical patient data, predefined targets, and safety thresholds, forming an ongoing feedback stream within the adaptive control loop.

Determine Need for Adjustment (Step 404). In Step 404, the adaptive control system evaluates whether a modification to the therapy parameters is required. If patient performance deviates from an expected trajectory or if a biometric parameter exceeds a predefined safety threshold, the system determines that an adjustment is necessary. For example, detection of excessive effort, incomplete motion, elevated heart rate, or reported pain may trigger a decision to increase assistance, reduce speed, shorten range of motion, or pause the exercise. Conversely, if the patient demonstrates stable vitals and successful task completion with minimal effort, the system may determine that assistance should be reduced or task difficulty increased. This decision logic supports an assist-as-needed control strategy.

Adjust Therapy Parameters in Real Time (Step 405). Upon determining that an adjustment is warranted, the system proceeds to Step 405, in which the adaptive control system 250 modifies one or more therapy parameters in real time. Adjustments may include changing applied torque or force, altering movement trajectories, modifying motion speed, adjusting compliance characteristics, or inserting rest intervals. These control updates are applied continuously or at defined intervals, enabling fine-grained, responsive assistance throughout the exercise session. The adaptive loop formed by Steps 403–405 repeats iteratively during the therapy activity.

Provide Patient Feedback and Perform Safety Checks (Step 406). In Step 406, the system provides real-time feedback to the patient regarding progress and performance, such as encouragement, status updates, or alerts displayed on the user-facing display 150 or delivered audibly. Concurrently, the system performs continuous safety monitoring, including checks for abnormal biometric readings, unexpected resistance, or hardware faults. If a safety condition is detected, such as excessive pain, unstable vital signs, or a patient-initiated stop request, the system temporarily interrupts the adaptive loop and transitions to a safe state, such as pausing or stopping the exercise.

Iterate or Advance Exercise (Step 407). In Step 407, the system determines whether to continue the current exercise, repeat the adaptive loop for additional repetitions, or advance to a subsequent exercise. Over multiple repetitions or sessions, the adaptive control system may implement progression logic, increasing task difficulty or reducing assistance as the patient’s capabilities improve. This iterative progression enables the rehabilitation protocol to evolve dynamically in response to measured patient performance and recovery trends.

Complete Session and Log Data (Step 408). When the prescribed exercise or therapy session is complete, the system executes Step 408 by terminating the adaptive control loop. The cobot returns the patient’s limb to a safe resting position and provides a session summary to the patient. All session data, including biometric trends, performance metrics, and applied control adjustments, are recorded and stored. The logged data may be transmitted to the predictive analytics module 270 and incorporated into the patient’s clinical record.

Throughout Steps 401–408, the adaptive control process operates in accordance with predefined clinical guidelines and safety constraints. Acceptable ranges for biometric parameters, exertion levels, and motion limits may be defined by clinicians and enforced by the control logic. The closed-loop adaptive approach enables the system to respond immediately to changes in patient condition, providing objective, data-driven adjustments that enhance safety, engagement, and therapeutic effectiveness.

In summary, FIG. 4 illustrates a closed-loop adaptive control methodology comprising sensing, evaluation, decision-making, and real-time actuation. This control strategy enables personalized rehabilitation assistance that continuously adapts to the patient’s evolving capabilities while prioritizing safety and clinical compliance.

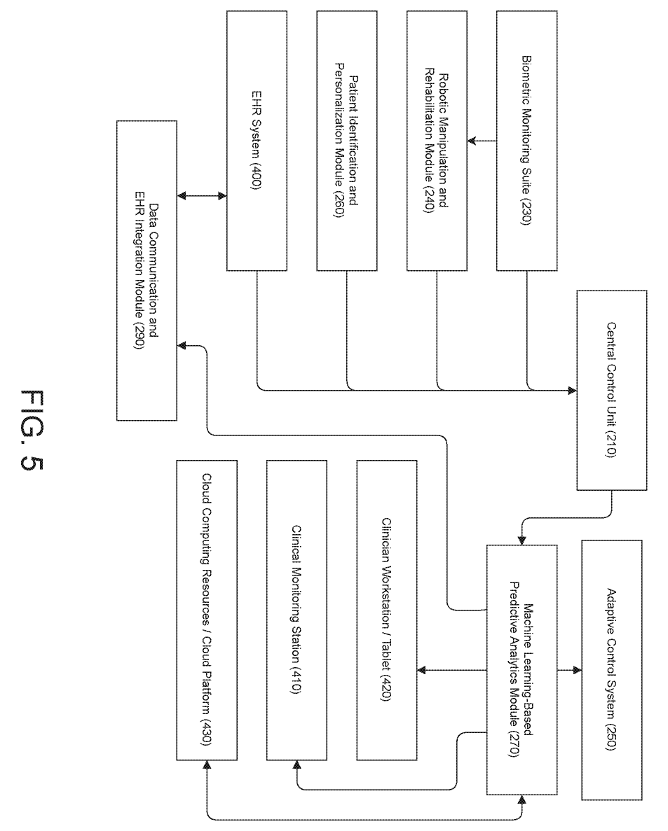

FIG. 5 provides a schematic representation of the predictive analytics module 270 of the cobot system, which uses machine learning (ML) and data analysis to forecast patient recovery and detect potential complications. The schematic illustrates how patient data flows into a predictive model and how the outputs of that model inform clinical decisions or system adjustments.

On the left side of FIG. 5, various data inputs are shown feeding into the predictive analytics module 270. These inputs include the time-series biometric data collected by the system (heart rate trends, blood pressure trends, respiratory rate, temperature over time, etc.), rehabilitation performance data (such as range of motion achieved, number of repetitions per session, level of assistance required, and patient compliance with exercises), and any relevant patient-specific information (age, type of surgery, medical history, baseline functional status). The module may also integrate external data from the patient’s electronic health records, for example, lab results or doctors’ notes, to enrich its analysis. All these data are aggregated in a feature set that represents the patient’s recovery trajectory so far.

At the core of FIG. 5 is a machine learning engine (depicted perhaps as a neural network or another model type) that processes the input data. This model could be trained on large datasets of previous patients who have undergone similar surgeries or rehab, enabling it to recognize patterns that precede certain outcomes. For instance, the model might have learned correlations such as: a sustained elevation in resting heart rate combined with reduced mobility could predict an oncoming infection or complication, or that patients whose range of motion improves by a certain rate per day are on track for a full recovery by a typical timeframe. The predictive analytics engine in this invention is designed to analyze biometric data to predict patient recovery trajectories and proactively identify complications. In practice, this could mean it outputs a prediction like, “Estimated time to achieve 90° knee flexion is 2 weeks based on current progress,” or “High risk of postoperative pulmonary complication detected (e.g., possible pneumonia) given the trend in respiratory data.”

FIG. 5 likely outlines multiple components of the analytics module 270: one sub-component might be a Trend Analyzer that calculates slopes or changes in key metrics (for example, improvement in walking speed per week, or change in pain levels over time), another might be a Risk Predictor that computes risk scores for defined complications (such as risk of readmission, risk of fall, etc.), and another could be a Recovery Progress Classifier that categorizes whether the patient is ahead of, on track with, or behind expected recovery milestones. These components together feed into the overall ML model or decision logic.

Importantly, the output of the predictive analytics module 270 is not just for academic interest, it directly feeds back into patient care. The right side of FIG. 5 illustrates how the module’s outputs are used. One output path is alerts/recommendations to clinicians: if the system predicts a complication or detects an anomaly, it can generate an alert for the medical staff. For example, if the model finds that the patient’s likelihood of developing a deep vein thrombosis (DVT) is high (perhaps due to low mobility and certain vital sign changes), it can flag this so doctors can intervene early. Another output is adjustment suggestions for the rehabilitation plan: the module can recommend changes such as modifying the exercise regimen or intensity. In fact, the system can automatically feed some of these recommendations into the adaptive control module (FIG. 4) as updated parameters, for instance, if the predictive model suggests the patient would benefit from more range-of-motion exercises to prevent stiffness, the next sessions can be adjusted accordingly. This is how the module recommends adjustments in patient care plans based on its analysis.

The schematic also indicates that the predictive module undergoes continuous learning. As new data comes in from each session, the module updates the patient’s profile. If an outcome occurs (say the patient did develop a certain complication), the system can incorporate that result to refine future predictions (this can be part of a feedback loop in FIG. 5). In some embodiments, the ML model can be retrained periodically on a cloud server 430 with accumulated data from many patients to improve its general accuracy. The local system might receive model updates or it might do on-the-fly learning for personalization. For example, the model might learn the patient’s unique recovery pace and adjust its trajectory predictions accordingly.

From a technical standpoint, the predictive analytics module 270 may employ algorithms such as regression analysis for trajectory prediction, classification models for risk detection (like logistic regression or random forests to classify risk level), or even advanced deep learning models that take sequences of time-stamped data as input (recurrent neural networks or transformers). The exact techniques are not critical to enabling the invention, as a skilled practitioner can choose an appropriate method. What FIG. 5 emphasizes is the functional role: using historical and current sensor data to anticipate future states in the patient’s recovery.

An example use-case of FIG. 5: Suppose a patient is recovering from knee surgery. The cobot has been collecting data for a week. The predictive module analyzes the trend that the patient’s knee flexion angle increased from 30° to 50° in the first week with a certain assistance level, and correlates that with patterns learned from past patients. It predicts that at this rate, the patient will reach 90° in another two weeks (which might be on schedule or slow, depending on the norm). It also notices the patient’s resting heart rate has been creeping up and they have had occasional spikes in temperature at night. This pattern, combined with slight drops in blood oxygen levels, triggers a prediction of a possible mild pulmonary complication. The system then raises an alert to the care team: “Predictive Analysis: Signs consistent with developing respiratory issue; recommend evaluation for infection or pulmonary function.” At the same time, it might adjust the rehabilitation to incorporate more breathing exercises or to hold off increasing intensity until the issue is addressed. By doing so, the system acts as an early-warning and decision-support tool, not only executing the current regimen but also looking ahead to ensure long-term success.

Furthermore, the predictive analytics module 270 contributes to personalization. If the model predicts a slower recovery trajectory for this patient (perhaps due to age or comorbidities captured in the data), the care plan can be personalized—maybe scheduling additional sessions or extending the rehabilitation period. These decisions are traditionally made by human clinicians, but FIG. 5 shows how the cobot can provide data-driven guidance to support those decisions. The system’s predictive capability thus embodies a form of artificial intelligence augmenting the healthcare process.

In summary, FIG. 5 highlights that the invention includes an intelligent analytics component which mines patient data for foresight. This ensures that the system is not merely reactive (adjusting in the moment), but also proactive in managing the patient’s recovery. It aligns with modern trends in healthcare where machine learning models are used to predict postoperative outcomes and complications, demonstrating a feasible and promising approach to improving patient care. By incorporating this into the cobot system, the invention provides a continuum from sensing and action (in earlier figures) to high-level insight and planning, thereby fully supporting patients and clinicians in the postoperative rehabilitation journey.

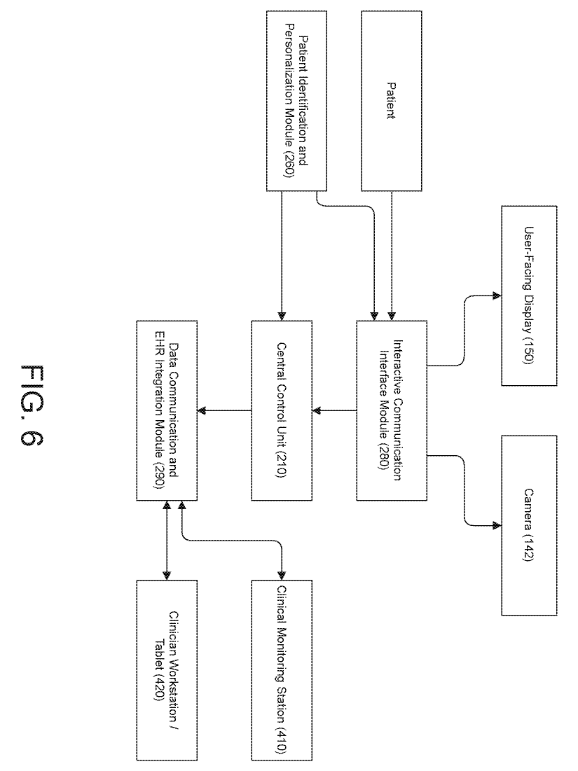

FIG. 6 illustrates the interactive communication interface 280 of the cobot system, highlighting how the cobot engages with the patient and integrates with the care team from a user interaction perspective. In this figure, one can see the various modes of communication the system supports and the user interface elements that facilitate these interactions.

At the center of FIG. 6 is the cobot’s display screen 150 which serves as the primary visual interface. The screen can present a graphical user interface (GUI) with multiple panels or modes. For instance, FIG. 6A might show the interface in “exercise coaching” mode: the screen displays an animated guide or avatar demonstrating a rehab exercise, alongside real-time data like repetition count and current heart rate. Large, easy-to-read text and icons are used so that patients of varying acuity (including elderly patients with impaired vision) can comfortably follow along. The interface may use color coding to convey status (green for safe ranges, yellow for caution, red for stop, etc., in terms of vital signs or performance). The patient can also see progress indicators, e.g., a progress bar for how many exercises are left in the session, or a gauge showing percentage of target range-of-motion achieved. All of this visual feedback is user-friendly and oriented towards encouraging the patient, as well as keeping them informed about their health status.

Accompanying the visual display 150, FIG. 6 also depicts audio communication elements. The cobot is equipped with speakers (positioned near the display 150 or head of the robot) that output synthesized speech and sounds. The system’s voice is polite and clear, giving instructions (“Lift your arm a bit higher… good job!”) or health updates (“Your heart rate is 80 beats per minute, which is within normal range”). The tone can be adjusted to be friendly and reassuring. The figure might illustrate sound waves emanating from the robot to indicate speaking. There are also microphones on the cobot that capture the patient’s voice input. For example, the patient can ask the cobot questions or report how they feel (“I’m feeling tired”) and the system’s voice recognition (if enabled) will interpret this and respond appropriately. Voice commands like “stop” or “help” could be configured for safety, so the patient always has a way to communicate distress.

Another key part of the interface in FIG. 6 is the touch input. The display 150 may be a touchscreen, allowing patients to press on-screen buttons. FIG. 6B might show an example menu on the touch UI: options such as “Start Exercise,” “Pain Level” (where the patient can tap a scale to rate their pain), or “Call Nurse.” The “Call Nurse” button is especially important, if the patient needs human assistance or has a question, tapping that will alert a nurse either through the hospital system or directly initiate a video call on the screen. This functionality ties into the telepresence capability: as indicated in the figure, upon requesting help, the interface can switch to a video call mode (perhaps shown as a window where the nurse’s face appears on screen and the cobot’s camera 142 transmits the patient’s image to the nurse). The nurse or doctor can speak with the patient through the cobot as if using a telehealth station. Because the cobot can move and orient its camera 142, the remote provider can even request different views (e.g., “please show me the surgical incision site”), and the cobot can adjust accordingly. FIG. 6C might illustrate a scenario of a family member or clinician communicating remotely, emphasizing that the cobot serves as a communication conduit.

The interface also provides haptic feedback in some cases. For example, the robotic arm 130 might have a soft pad or a haptic device that can gently vibrate or tap the patient’s arm to get their attention if they are not looking at the screen. Alternatively, if the patient has low vision, the system could vibrate to signal the start of an exercise or use auditory cues primarily. The design is meant to be multi-sensory to accommodate different needs. FIG. 6 might denote a haptic output with an icon or waveform near the patient’s arm, indicating the cobot touching or tapping lightly as part of interaction.

An important aspect shown in FIG. 6 is personalization of the interface. The system can adapt its communication style to the patient’s preferences. For instance, if a patient does not speak the default language, the system can switch languages for both text and voice output. If a patient prefers more detailed explanations about their exercises or condition, the system can provide that; whereas another patient might prefer minimal information and more encouragement, which the system can adjust to. These preferences might be stored in the patient profile (from the identification module 260). The interface could also have different themes, e.g., a playful, cartoon-like interface for pediatric patients versus a more formal interface for adults. The aggregated hardware allows various forms and visual changes to appearance to cater to patient comfort, which extends to the digital interface as well (colors, avatar styles, etc., can be matched to patient age or mood).

Another panel likely shown in FIG. 6 is the clinician interface view. While much of the interface is for the patient, some aspects are for clinicians. For example, when a nurse comes into the room, they might use the cobot’s display 150 to quickly check the patient’s stats or to input notes. The cobot could have a mode where it displays a summary of the latest vitals, wound check, and exercise performance, possibly protected by authentication (the nurse might scan an ID badge or enter a PIN on the cobot to unlock that view). FIG. 6 might show an example of a clinician dashboard on the cobot’s screen with more detailed medical data and controls to adjust settings (like turning certain monitoring on/off, or acknowledging alerts).

In terms of system architecture, the communication interface module 280 in FIG. 6 is also responsible for wireless communications like sending messages or data to other devices. So the figure could include icons for Wi-Fi or Bluetooth connectivity, indicating that the cobot is continuously communicating with backend systems. For instance, as the patient interacts, data might be sent simultaneously to the nurse’s tablet 420 or the hospital monitoring station 410. A symbol of a cloud or database might appear to denote data syncing to the central record (which ties into FIG. 7).

In summary, FIG. 6 encapsulates the human-machine interaction layer of the system. The cobot behaves not just as a machine executing tasks, but as an interactive assistant that communicates naturally and effectively with the patient. By providing visual, auditory, and haptic feedback, and by enabling remote human communication channels, the interface ensures that the patient is never isolated in their care. The design follows medical device usability standards, aiming to be intuitive even under stress or for users with impairments. This interactive interface is crucial for patient acceptance of the technology and for integrating the cobot into the healthcare workflow as a helpful teammate for both patients and providers.

FIG. 7 is a communication diagram illustrating how the cobot-based system connects and exchanges data with external healthcare information systems, particularly the hospital’s electronic health record (EHR) system 400. This figure highlights the flow of information between the cobot and the digital infrastructure of patient care, ensuring seamless documentation, remote monitoring, and integration into clinical workflows.

In FIG. 7, the cobot system 100 is shown on one side, while on the other side are representations of external systems: an EHR 400 server/database, a clinical monitoring station 410, and possibly a secure cloud service 430. Arrows between the cobot and these entities depict data transfer pathways.

One primary pathway is the EHR integration link 290. As indicated, the cobot transmits patient data (vital signs, exercise logs, alerts, etc.) to the hospital’s EHR 400 in a secure, periodic or real-time manner. For example, after each rehab session, the cobot can automatically upload a summary of the session (e.g., “10am session: patient completed 10 knee bends with 20% assistance, heart rate 80-100 bpm, no adverse events”) to the EHR 400 as a note or structured data entry. Vital signs measured by the cobot can populate the flowsheets in the patient’s chart just as if a nurse had entered them manually. FIG. 7 might label this as “Biometric Data Upload” with an arrow from the cobot to the EHR 400. The data format and protocol could utilize HL7 or FHIR standards for compatibility with existing systems, but those details are abstracted in the diagram.

Another pathway is for remote monitoring by clinicians. The figure could show a nurse’s workstation 410 or a physician’s tablet 420 receiving live data from the cobot. For instance, in the ICU or post-surgical ward, nurses often have a central station where they can see patients’ vital signs. The cobot essentially acts as a vital sign monitor and therapy device in one, so it streams pertinent data to those stations. If the patient’s blood pressure goes out of range or if the cobot issues an alert (like “patient skipped exercise due to pain”), that information is sent to the nurse’s console. FIG. 7 indicates this with a “Real-time Alert” arrow, e.g., the cobot sends an alert to the nurse’s pager or the central system if thresholds are crossed. This integration ensures that the clinical oversight remains in place: even though the cobot operates autonomously, the human care team is kept in the loop via the digital health network.

Security and privacy are paramount in these communications. The figure explicitly notes that all transmitted data is encrypted and sent over secure channels (illustrated perhaps by a lock icon on the communication arrows). The system uses the hospital’s secure Wi-Fi or a VPN tunnel if over the internet, complying with regulations like HIPAA. As mentioned earlier, the data is stored in HIPAA-compliant cloud 430 or network storage. FIG. 7 could depict this as the data passing through a “Secure Gateway” before reaching the EHR 400. This ensures patient personal information and health metrics are protected during transmission and storage.

FIG. 7 also shows bidirectional communication in some cases. Not only does the cobot send data out, but it can also receive data or commands from external systems. For example, a physician updating the rehab orders in the EHR 400 (like changing the exercise schedule or adding a note “allow more rest if pain > 5”) could be fetched by the cobot so that it updates its operation accordingly. Similarly, if there is an enterprise scheduling system, the cobot might receive a message like “Patient scheduled for physical therapy at 3pm” and it can prepare or go to the patient at that time. This two-way communication ensures the cobot is synchronized with the hospital’s workflows and any changes in the patient’s care plan.

Another element that may appear in FIG. 7 is integration with other devices or systems, such as the pharmacy or lab systems. For instance, the diagram could show a link from the cobot to a pharmacy system if the cobot needs to verify a medication before helping administer it (recall earlier mention that the cobot could potentially fetch medications or inject pain relief under supervision). Or integration with a lab system if the cobot takes a blood sample and needs to label it in the records. While these are peripheral to the core rehab monitoring function, including them in the diagram emphasizes the cobot’s role as a connected device in the healthcare Internet of Things (IoT).

The data logging and compliance aspect is also illustrated. Every interaction and data point the cobot handles can be logged for compliance and audit purposes. FIG. 7 might show a storage icon where logs are kept (either locally on the cobot with backup to the server). This log would include timestamps of all measurements, adjustments made by the adaptive control, when alerts were triggered and to whom they were sent, and confirmations that data was successfully written to the EHR 400. Having this log helps in quality control and meeting documentation standards; for example, it provides evidence that vital signs were monitored and recorded appropriately.

In practice, by interfacing with the EHR 400, the system enables efficient documentation and continuity of care. Nurses and doctors can review the automatically updated chart and see trends. It reduces manual data entry, freeing up staff time. Moreover, trends stored in the EHR 400 can be reviewed alongside other clinical data (like lab results or clinician observations) to get a holistic picture of the patient. If the patient is transferred or seen by another department, all data collected by the cobot travels with them through the EHR 400.

Finally, FIG. 7 underscores remote access: authorized clinicians could remotely access the cobot’s data (through the hospital network or a secure cloud 430). For example, a physical therapist could log in from a tablet or computer 420 in their office to see how the patient did in the morning session, then decide if afternoon therapy is needed. Or a surgeon could check the recovery progress indicators tracked by the cobot to make postoperative decisions. The connectivity thus supports telemedicine aspects; in some embodiments, a central hub might monitor multiple such cobots across different rooms or even different facilities.

To sum up, FIG. 7 shows that the cobot system is not an isolated tool but part of an integrated health IT system. By wirelessly communicating with EHR 400 and other clinical systems, it ensures that all stakeholders have the necessary information and that the patient’s data flows into the official medical record in real time. This integration also facilitates compliance with healthcare regulations and protocols, as everything the cobot does is documented just like human-provided care would be. The result is a more connected, transparent, and accountable system of care where the cobot augments the healthcare team effectively.

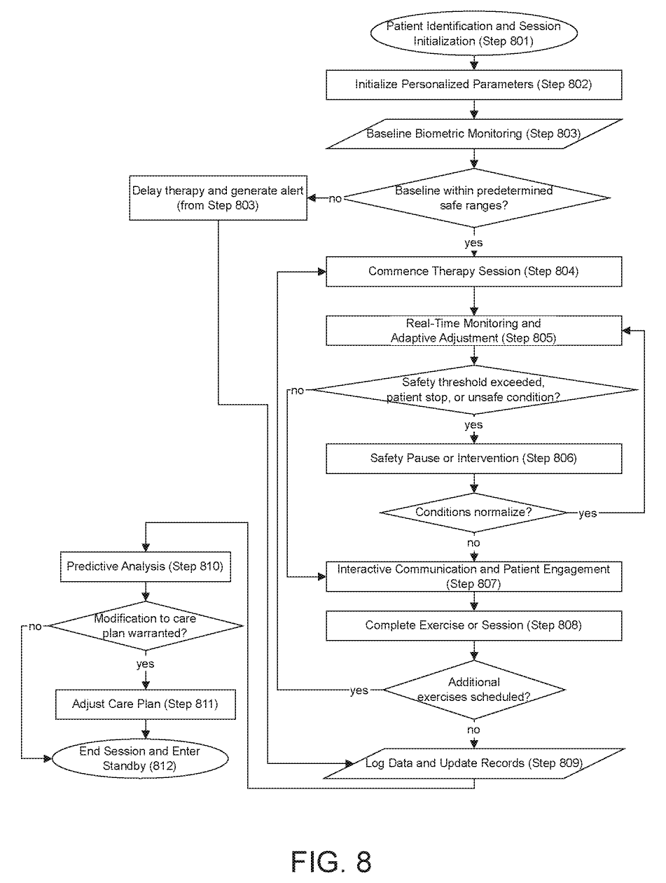

FIG. 8 is a flowchart depicting an overall method of operation of a cobot-based patient care system. The method integrates the processes described with respect to FIGS. 1–7 into a unified, step-by-step workflow, illustrating how system hardware and software components cooperate to deliver personalized patient care from initial engagement through session completion, data logging, and preparation for subsequent sessions. The method of FIG. 8 supports the method aspects of the invention and proceeds through the following numbered steps.

Patient Identification and Session Initialization (Step 801). The method begins when the cobot engages with or is assigned to a patient. The system identifies the patient using one or more identification mechanisms, such as reading an RFID tag, scanning a barcode, or performing facial recognition using the camera 142. Upon identification, the cobot may greet the patient and confirm identifying information. The system retrieves patient-specific data, including care plans, clinician instructions, and historical records, from local memory and/or the electronic health record system 400.

Initialize Personalized Parameters (Step 802). The system initializes session parameters based on the identified patient. This includes loading physiological baseline ranges, therapy schedules, safety constraints, movement restrictions, and personalization settings. Where applicable, the system performs calibration routines, such as determining limb dimensions, motion limits, or baseline strength levels, to tailor subsequent therapy actions to the patient.

Baseline Biometric Monitoring (Step 803). Before active intervention, the system acquires baseline biometric measurements using the biometric monitoring suite 230. Measurements may include heart rate, blood pressure, respiratory rate, oxygen saturation, temperature, and related physiological indicators. The system evaluates whether baseline values fall within predetermined safe ranges. If one or more parameters are outside acceptable limits, the system may delay therapy and generate an alert. If baseline conditions are acceptable, the method proceeds.

Commence Therapy Session (Step 804). The cobot initiates the rehabilitation or care session. The system provides instructions or demonstrations to the patient via the interactive communication interface and the user-facing display 150. The robotic manipulator 130 is positioned to assist, guide, or resist patient movement as appropriate. Initial assistance or resistance levels are set based on the patient profile, and the system begins execution of the prescribed exercise, therapeutic activity, or care intervention.

Real-Time Monitoring and Adaptive Adjustment (Step 805). During execution of the session, the system continuously monitors biometric signals and performance metrics. Sensor data are supplied to the control unit 210 and adaptive control system 250. The system evaluates patient effort, movement quality, repetition counts, and physiological response in real time and adaptively adjusts therapy parameters as needed, such as modifying force, speed, range of motion, or assistance level. This step corresponds to the adaptive control loop described in FIG. 4.

Safety Pause or Intervention (Step 806). If a safety threshold is exceeded, a patient-initiated stop is detected, or an unsafe condition arises, the system temporarily pauses or terminates the active intervention. The cobot provides guidance or reassurance to the patient and may summon clinical assistance or transition the system to a safe state. If conditions normalize, the system may resume the session by returning to Step 805.

Interactive Communication and Patient Engagement (Step 807). The system engages the patient through interactive communication during the session. The cobot provides progress indicators, encouragement, and prompts via visual or audio output, and may solicit patient feedback, such as pain levels or fatigue. The system may also facilitate real-time communication with a remote clinician via telepresence, as described with respect to FIG. 6.

Complete Exercise or Session (Step 808). Upon completion of the prescribed exercise or therapy session, the system transitions the patient to a safe resting state, disengages the robotic manipulator 130 as appropriate, and provides session-completion feedback. If multiple exercises are scheduled, Steps 804–807 may be repeated iteratively before concluding the session.

Log Data and Update Records (Step 809). The system compiles session data, including biometric trends, adaptive control actions, patient responses, and notable events. The data are transmitted to the electronic health record system 400 via the data communication and EHR integration module 290, ensuring that clinicians have access to up-to-date records of patient activity and system-assisted care.

Predictive Analysis (Step 810). Session data are supplied to the predictive analytics module 270. The module evaluates patient progress relative to historical data and predictive recovery models, generating updated forecasts, risk assessments, or recommendations.

Adjust Care Plan (Step 811). If predictive analysis indicates that a modification to the patient’s care plan is warranted, the system adjusts future therapy parameters, schedules, or exercises and/or generates recommendations or alerts for clinician review. If no adjustment is required, the existing plan is maintained.

End Session and Enter Standby (Step 812). The cobot concludes the session and transitions to a standby or reassignment state. The system confirms patient safety, optionally continues passive monitoring, and prepares for subsequent tasks or sessions.

Throughout execution of Steps 801–812, safety and compliance protocols operate continuously in parallel. The system enforces clinical limits, collision avoidance, secure data handling, and fault detection. If any fault or deterioration in patient condition is detected at any step, the system transitions to a safe state and involves human caregivers as needed.

Overall, FIG. 8 illustrates a comprehensive method for operating a cobot-based patient care system, encompassing patient identification, monitoring, adaptive intervention, communication, data integration, and predictive planning. The method may be implemented as software instructions executed by the control unit 210 and stored on a non-transitory computer-readable medium, thereby supporting system, method, and computer program product embodiments of the invention.

In conclusion, the Detailed Description above, with reference to FIGS. 1-8, provides a full and enabling disclosure of the Cobot-Based Patient Care for Biometric Monitoring and Postoperative Rehabilitation Support system. The system embodiment (FIG. 1-3) and its architecture (FIG. 2) detail the physical and logical components that fulfill all the functionalities outlined in the independent system claim, including the mobile platform, sensor suite, robotic rehabilitation means, adaptive control, identification, machine learning analytics, communication interface, EHR connectivity, and safety features. The method of operation (FIG. 8 and related flowcharts in FIG. 4) describes step-by-step how the system is used in practice, supporting an independent method claim by delineating the interactions between patient and cobot and the adjustments made in real time. Finally, the description inherently supports an independent claim directed to a non-transitory computer-readable medium, by disclosing that the various control and analysis functions are carried out by software instructions executed on computing hardware within the system. Alternative embodiments, including non-humanoid configurations and different deployment scenarios, have been described to illustrate that the invention can be modified without departing from its essential scope, ensuring that those skilled in the art can make and use the invention as claimed. Each figure and the corresponding description above contribute to a comprehensive understanding of how the cobot-based system improves patient care in the postoperative setting by combining robotics, biometric monitoring, adaptive control, and intelligent data integration in a novel and non-obvious way.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A collaborative robot for postoperative patient care, comprising:
a mobile platform configured to autonomously navigate to a patient’s bedside;
an integrated biometric monitoring suite comprising sensors including at least one of a heart rate sensor, a respiratory rate sensor, a temperature sensor, a blood pressure sensor, and a motion tracking sensor, the biometric monitoring suite being configured to continuously monitor patient vital signs and motion;
a robotic rehabilitation module including a robotic manipulator configured to assist the patient with physical therapy exercises, wherein an assistance level provided by the robotic manipulator is adjustable;
a control unit operatively coupled to the mobile platform, the biometric monitoring suite, and the robotic rehabilitation module, the control unit configured to modify operation of the robotic rehabilitation module in real time based on patient-specific biometric feedback from the biometric monitoring suite;
a patient identification module configured to identify the patient and retrieve an individualized care plan for the patient;
a wireless communication interface configured to transmit patient data collected by the biometric monitoring suite to a remote healthcare information system;
an interactive patient communication interface comprising at least one of a visual display, a speaker, or a haptic feedback device to provide the patient with exercise instructions and progress updates; and
one or more safety systems including at least one collision detection sensor and a privacy safeguard configured to restrict access to patient data.

2. The collaborative robot of claim 1, wherein the mobile platform comprises an autonomous navigation system including one or more navigation sensors for obstacle detection and control software for obstacle avoidance and precise bedside positioning.

3. The collaborative robot of claim 1, wherein the biometric monitoring suite further comprises a motion-tracking subsystem including at least one accelerometer configured to monitor the patient’s movements and posture during rehabilitation exercises.

4. The collaborative robot of claim 1, wherein the robotic manipulator comprises an articulated robotic arm configured to perform guided range-of-motion exercises with the patient’s limbs and to apply an adjustable resistive or assistive force to the patient’s movements.

5. The collaborative robot of claim 1, wherein the control unit is configured to adjust the assistance level provided by the robotic rehabilitation module by increasing the assistance when the patient’s biometric feedback indicates fatigue or difficulty and decreasing the assistance as the patient’s measured strength or range of motion improves over time.

6. The collaborative robot of claim 1, wherein the control unit comprises a real-time adaptive control system that continuously receives data from the biometric monitoring suite and dynamically updates one or more therapy parameters in response to changes in the patient’s biometric signals.

7. The collaborative robot of claim 1, wherein the patient identification module comprises a radio-frequency identification reader and a camera with facial recognition software for verifying the patient’s identity and customizing operation of the collaborative robot according to the patient’s individualized care plan.

8. The collaborative robot of claim 1, wherein the control unit includes a machine learning-based predictive analytics module configured to analyze data from the biometric monitoring suite to predict the patient’s recovery trajectory, identify potential complications, and recommend adjustments to the patient’s care plan.

9. The collaborative robot of claim 1, wherein the wireless communication interface is configured to communicate with an electronic health record system to send real-time updates of the patient’s biometric data and rehabilitation progress and to receive healthcare provider input or updated care instructions from the electronic health record system.

10. The collaborative robot of claim 1, wherein the one or more safety systems include a data encryption module to protect patient information and predefined operational constraints to ensure the collaborative robot operates in accordance with applicable medical device regulations.

11. A method for providing biometric monitoring and postoperative rehabilitation support to a patient using a collaborative robot, the method comprising:
moving the collaborative robot autonomously to a position proximate to the patient’s bedside while using safety sensors to detect and avoid obstacles;
identifying the patient using a patient identification module of the collaborative robot to retrieve an individualized care plan for the patient;
monitoring the patient’s vital signs and motion via the biometric monitoring suite on the collaborative robot, the vital signs including at least one of heart rate, respiratory rate, body temperature, and blood pressure;
assisting the patient in performing physical therapy exercises with a robotic manipulator of the collaborative robot, wherein an assistance level provided by the robotic manipulator is adjusted during the exercises;
adjusting, by the control unit of the collaborative robot, the assistance level in real time based on biometric feedback from the biometric monitoring suite indicating the patient’s performance or fatigue;
providing instructional guidance and feedback to the patient about the exercises via the interactive patient communication interface of the collaborative robot;
analyzing data collected from the biometric monitoring suite using a machine learning-based predictive model to forecast the patient’s recovery trajectory and to detect potential complications;
transmitting the patient’s biometric data and rehabilitation progress data to a remote healthcare information system via the wireless communication interface; and
operating the collaborative robot in accordance with safety and privacy protocols, including detecting and avoiding any impending collisions and safeguarding the patient’s personal data during the method.

12. The method of claim 11, wherein identifying the patient comprises using the patient identification module to scan a radio-frequency identification tag associated with the patient to confirm the patient’s identity and retrieve the corresponding care plan.

13. The method of claim 11, wherein monitoring the patient’s vital signs comprises continuously measuring the patient’s heart rate, respiratory rate, body temperature, and blood pressure with the biometric monitoring suite, and generating an alert if any of the measured vital signs falls outside a predetermined safe range.

14. The method of claim 11, wherein assisting the patient in performing physical therapy exercises comprises using the robotic manipulator to guide a limb of the patient through a prescribed range-of-motion exercise while providing an adjustable assistive or resistive force to the limb.

15. The method of claim 11, wherein adjusting the assistance level in real time comprises increasing the assistance when the patient’s biometric feedback indicates onset of fatigue or difficulty performing the exercise, and decreasing the assistance as the patient’s strength and performance improve over multiple sessions.

16. The method of claim 11, wherein analyzing the data using the machine learning-based predictive model comprises comparing the patient’s current biometric trends and exercise performance to historical recovery data in order to estimate a recovery time frame and to identify any deviation from an expected recovery trajectory.

17. A non-transitory computer-readable storage medium storing instructions that, when executed by one or more processors of a collaborative robotic system for postoperative patient care, cause the system to:
move autonomously to a patient’s bedside while utilizing one or more safety sensors to detect and avoid obstacles;
identify the patient via a patient identification module and retrieve an individualized care plan for the patient;
monitor the patient’s biometric data, including at least heart rate, respiratory rate, body temperature, blood pressure, and motion, using the collaborative robotic system’s biometric monitoring suite;
assist the patient in performing physical therapy exercises with a robotic manipulator, including adjusting a level of assistance provided to the patient based on real-time biometric feedback;
provide interactive guidance and feedback to the patient during the exercises through a communication interface on the collaborative robotic system;
analyze the patient’s biometric data using a machine learning algorithm to predict the patient’s recovery progress and to detect potential health risks; and
transmit data regarding the patient’s vital signs and rehabilitation progress to a remote healthcare information system.

18. The non-transitory computer-readable storage medium of claim 17, wherein the instructions to identify the patient include instructions to capture an image of the patient with a camera on the collaborative robotic system and to perform facial recognition on the captured image.

19. The non-transitory computer-readable storage medium of claim 17, wherein the instructions to analyze the patient’s biometric data include instructions to apply a trained machine learning model that outputs an indication of whether the patient’s recovery is on track and generates an alert if an anomaly is detected in the patient’s vital sign patterns.

20. The non-transitory computer-readable storage medium of claim 17, wherein the instructions further cause the collaborative robotic system to halt movement upon detecting an obstacle via the one or more safety sensors, thereby preventing a collision with the patient or any surrounding object.
